(19) United States
(12) Patent Application Publication
(10) Pub. No.: US 2004/0092901 A1
(43) Pub. Date: May 13, 2004

(54) Diaper changing kit

(76) Inventors: Ronald Wayne Reece, Marysville, OH (US); Raymond George Newell, Marion, OH (US)

Correspondence Address: Sean M. Casey Co., L.P.A., Attention: Sean M. Casey, P.O. Box 710, New Albany, OH, 43054-0710, US

(21) Appl. No.: 10/292,318

(22) Filed: Nov. 11, 2002

(51) Int. Cl. A61F 13/15; A61F 13/20

(52) U.S. Cl. 604/385.06

(57) ABSTRACT

A self-contained diaper changing kit that can include a diaper that is formed with interior and exterior surfaces of anterior and posterior portions, and which incorporates a generally unitary container that is configured to attach to the exterior surface of the diaper. The container is formed with a plurality such as three or more unsealable and also possibly resealable receptacles. One or any of the receptacles can be further adapted with at least one scission element that is configured in any number of arrangements to unseal the corresponding receptacle. In one variation of many possible configurations, the container can include at least three unsealable and possibly resealable receptacles wherein one receptacle can contain a substantially liquid substance such as a lotion or medicament, another receptacle can be received with a substantially powdered substance, and another receptacle can capture and store a sheet material such as a pre-dampened towel or wiping material. Any of the receptacles can further be adapted to releasably capture one or more sealed packets that can be filled with a substantially flowable substance such as powder, lotions, and the like. In the resealable configurations, the scission elements formed about the receptacles can be further adapted with a severing tear strip, a severing rip cord having a distal pull tab, and or as a flexible and resealable tongue and groove sliding-type zipper like fastener.

### TECHNICAL FIELD

This invention relates to a diaper changing kit. This diaper changing kit contains an integrally assembled diaper and unitary storage and dispensing chamber having a plurality of the lotions, oils, powders, wipes, disposal bag, gloves, and or packets thereof and the like that are useful or desirable for the process of changing the diaper. These elements are arranged so that all of these materials are within easy, convenient reach of the person changing the diaper while being substantially out of the reach of the child or other person wearing the diaper.

### BACKGROUND OF THE INVENTION

Human babies and very young children, and older persons suffering from incontinence and individuals afflicted with other maladies are prone to excrete various bodily fluids or to eliminate waste as needed and often without the ability to control the time, place, or convenience. As the term is used herein, a diaper or napkin or loincloth or similarly capable undergarment is defined to include a piece of absorbent material, such as elastomeric and polymeric materials, cellulosic materials such as paper, and fabric and cloth materials made therefrom and from cotton, wool, and various synthetic fibers. Such diapers or undergarments or napkins are further contemplated to be sized, shaped, and configured to be positioned between the legs of a person and fastened about the waist to contain bodily excretions and or waste of a baby, incontinent adult, or other person needing such extracorporeal containment.

Such contemplated diapers, loincloths, napkins (including sanitary napkins often donned by women during menstrual cycles, and by infants in the chiefly British nomenclature), and similarly capable undergarments can be washable and reusable or single-use and disposable and are frequently used to contain such excretions or bodily waste and to create a healthier, more sanitary environment for both the child or other diaper or napkin wearing person as well as for the attending caretaker(s), if any. A wide range of such diapers and napkins are available for this purpose. Diapers are made of cloths such as cotton, of natural and synthetic materials such as cellulosic and other polymeric materials, and of other substances and materials and of hybrids and combinations thereof.

Various types of fasteners are also typically used to fasten the diaper about the wearer and can include, for example, hook and loop fasteners, pins, adhesive strips, snaps, ties, and combinations thereof. Diapers may include additional features such as, for example, custom shapes that can be adapted for various wearing arrangements, and that can include elastomeric waist or leg bands, decorations that may appeal to children, or other beneficial or fanciful alterations. Diapers often are available in a range of possible configurations that establish various capabilities, capacities, absorbencies, and sizes.

Regardless of the specific type of diaper that a baby or other person wears, the diaper usually operates by absorbing, adsorbing, or otherwise containing solid or liquid excretions or waste that is eliminated. Once this waste has been eliminated and contained by the diaper, it is known to those skilled in the art to remove the soiled diaper for cleaning or disposal, and to replace it with a fresh diaper. For infants, this process is often known as changing the diaper, or colloquially as “changing the baby” in the American context, or “replacing the napkin” in various other cultures.

As can readily be appreciated by parents, grandparents, guardians, caretakers, and others with skill in the art, changing a diaper involves a plurality of steps and supplies. For example, it is desirable to have a waste container capable of receiving the soiled diaper. As a further example, it is common for the person that is changing the diaper, to use a cloth, rag, or other material to wipe any residual waste from the baby before applying the new diaper. It may also be desirable to employ powders, lotions, balms, oils, or the like for use on the wearer's, skin or on the skin of the person changing the diaper or napkin.

Such powders, lotions, oils, balms, diapers, receptacles, and other ancillary supplies can consume, collectively, a significant amount of shelf or counter space. To deal with this space limitation, those for whom changing a diaper is a common task will usually dedicate one or more areas of the home or care giving facility to the process of changing a diaper. In such an area, all of the diapers, receptacles, powders, lotions, ointments, and other required or desirable materials or supplies can be conveniently stored where they are readily accessible to the individual engaged in changing the diaper. This type of dedicated arrangement can increase the ease and efficiency of changing a diaper, but it does little to help the caregiver, parent, or guardian that must change diapers in other locations or during transit of the diaper wearing individual or infant.

To ease and to enable the process of changing diapers in such inconvenient places, many caregivers or parents have come to depend upon a dedicated mobile changing bag or case that is provisioned with all of the accoutrements needed for easy and sanitary diaper changes. Although there are various possibly desirable modifications and features, such a mobile bag or case can be a large, multi-compartmental bag with a plurality of pockets and often a shoulder strap. The caregiver or parent carries in the bag the various items such as fresh diapers, disposal or storage receptacles or bags for soiled items, lotions, balms, and other ancillary material used to change a diaper.

The mobile changing bag can also contain food, beverages, toys, and other baby related tools and items as well as articles that may be needed for older individuals that may need portable diaper changing items. The mobile changing bag is commonly used by many parents and caregivers, but there are limitations to its utility. A loaded mobile changing bag can be heavy, cumbersome, and uncomfortable to carry. It may be difficult to readily locate a given powder, lotion, or supply when needed. It may be especially difficult to hold a naked and squirming baby with one hand while with the other hand attempting to locate the needed container of ointment, powder, or other supply.

Those with skill in the art understand that these and other problems arise when attempting to change a diaper in an inconvenient location. Since it is usually necessary to take the baby out of the home or the diaper-wearing individual out of the care facility at least occasionally, the need exists for an easy and convenient means for changing a diaper outside of the home or other dedicated diaper changing location.

Some attempts have been made to overcome past limitations and have been introduced in various forms including diaper changing kits. Such kits generally include, in one container or kit, a diaper, a waste receptacle, and some of the lotions, powders, ointments, and the like that are used in the process of changing a diaper. For example, Feldman in U.S. Pat. No. 4,931,052 describes a storage container that is adapted to receive a wiping cloth. While it may be convenient to have such a wiping cloth close at hand while changing a baby, the person changing the diaper may require lotions, ointments, powders, or other accessory materials. This is a significant limitation, since the individual changing the diaper might have to accomplish the task at hand without the other supplies, thereby risking an uncomfortable or unsanitary state for the baby or older diaper wearer. Alternatively, the person changing the diaper might resort to searching through a mobile changing bag or the like for the additional materials, in which case the diaper changing kit has not reduced the already described burdens and limitations associated with the mobile changing bag.

Other diaper changing kits include not only the diaper and wiping cloth but also some of the lotions, powders, and the like that are absent in the '052 patent. One possible arrangement of such an inclusive kit is a bag or package that contains sub-compartments, each adapted to receive a diaper or some diaper related accessory. An example of this type of kit is suggested in U.S. Pat. No. 4,702,378 to Finkel et al., which is limited to rolling a diaper and a plurality of related items in a thin plastic film. Like the mobile changing bag, the Finkel et al. kit suggests a plurality of compartments, each able to receive and to contain one or more of the items needed to change a diaper. This style of kit, however, also shares some of the limitations of the mobile changing bag. Individual items may shift position or be separated from the '378 kit during transport and use. Even if each item of the '378 kit remains in its original position, it may be difficult to easily locate and remove or to open a desired item. The relatively small size of the individual lotions, ointments, powders, wipes, and the like compound such difficulties. Attempting to change a diaper in the back of a car, in a public restroom, or in some other similarly inconvenient location can further increase the attendant challenges.

Other arrangements of prior diaper changing kits attempt to overcome the problems associated with shifting or otherwise inaccessible supplies. Some of such kits contemplate the attachment of the various accessory items to the diaper itself. For example, one of the teachings of U.S. Pat. No. 6,004,307 to Colon et al. is restricted to attachment of a plurality of hygienic items to an exterior portion of a diaper. In U.S. Pat. No. 4,221,221 to Ehrlich, another example also suggests that the accessory items can be attached to the diaper. The '307 Colon kit further discloses the use of an outer wrapper that can be used as a baby-changing surface. Even with one or more of the Colon '307 suggestions, it still can be difficult for the person changing the diaper to easily locate each individual item, especially if these items are attached to the side, back, or bottom of the Colon '307 diaper. More significantly, it can still be awkward to open the individual items without both hands, which is the norm with infants where one hand is typically occupied restraining a baby or holding a diaper in place, or both.

What has long been needed in the art is a diaper changing kit that contains all of the most desirable items required to change a diaper, that conveniently packages each item in an easy to open and possibly resealable container, and that arranges these items such that each and every diaper changing related item can be conveniently reached in the normal process of changing a diaper. Even further, it would be a significant improvement over prior art devices and kits if each of these features could be implemented while also further preventing or limiting access by the diaper wearer. The present invention meets these and other needs, is widely compatible with diapers, and does not add any complexities to manufacture or use.

### SUMMARY OF INVENTION

In its most general configuration, the present invention advances the state of the art with a variety of new capabilities and overcomes many of the shortcomings of prior devices in new and novel ways. In one of the many preferable configurations, a self-contained diaper changing kit incorporates a diaper that has interior and exterior surfaces that are sandwiched to form a substantially anterior portion that is connected by an inferior perineal portion to a generally posterior portion.

Further included is a generally unitary and compartmentalized container that is attached proximate to the exterior surface of the anterior portion of the diaper and which is preferably formed with a plurality of unsealable receptacles wherein at least one receptacle of the plurality is formed with at least one scission element adapted to unseal the at least one receptacle to dispense the contents thereof. In variations of this arrangement, the generally unitary container can be configured to be releasably affixed to the exterior surface of the anterior portion of the diaper.

In additional variations of any of the embodiments according to the instant invention, the generally unitary container may also incorporate at least one of the plurality of unsealable receptacles to be filled and or received with a substantially flowable substance such as a powdered and or liquid lotion or medicament. In addition to or in an alternative configuration, the generally unitary container of the self-contained diaper changing kit can include at least one of the plurality of unsealable receptacles being adapted to contain and or containing a sealed packet or pouch that is adapted to be filled with a similarly substantially flowable substance. In this and other modifications, the generally unitary container may be further adapted to define at least one aperture that is formed in the container and that opens into a receptacle of the plurality that is configured to remain unsealed and that receives the sealed packet filled with the substantially flowable substance.

In further variations, the self-contained diaper changing kit is formed with the scission element of the generally unitary container being configured as a severing tear strip that can be grabbed with one hand and pulled to sever a portion of the respective unsealable receptacles to establish an opening through which the contents of the respective receptacle can be accessed. In an alternative or additional variation of any of the preceding embodiments and configurations, the scission element can also be formed as a severing rip cord having a distal pull tab that creates the opening into the respective receptacle. In even other desirable modifications and variations to any of the preceding embodiments and arrangements, the scission element can also be a substantially flexible and resealable tongue and groove sliding type fastener that can be incorporated to establish the opening into a respective one or more of the receptacle(s).

While many tongue and groove type fasteners are available on the market, those sold under the tradename and trademark commonly known to those skilled in the arts as the Zip-Loc® brand offer a variety of possibly desirable solutions to the scission elements contemplated here. In one contemplated variation of the tongue and groove type scission elements noted, a tongue and groove type scission element includes the type that is operable to be resealed wherein a thumb and a forefinger can be press together to sandwich the tongue and groove elements together as the thumb and forefinger slide from one end to an opposite end of the scission element so as to close the opening into the receptacle. In another variation of the instantly described Zip-Loc®-type scission element, a zipper-like slider component is interposed about the tongue and groove elements, which scission element opens and separates, and closes and joins the respective opening into the receptacle as the zipper-like slider is moved from one side to an opposite side of the scission element. In any of the contemplated embodiments, variations, arrangements, and modifications described herein, the scission element in any of its contemplated forms may be substantially rigid or flexible as may be suitable for various applications and desired capabilities.

Other variations of any of the preceding embodiments and configurations are also contemplated by the instant invention that are directed a self-contained diaper changing kit that can include, among other components and modifications, a diaper formed with an interior and an exterior surface and to have a substantially anterior portion that is connected to a generally posterior portion by an inferior perineal portion sized and shaped relative to the other elements to comfortably fit about the perineum region and between the legs of the person wearing the diaper. In this variation, a generally unitary container is also incorporated that is attached close or proximate to the exterior surface of the anterior portion, although other locations may be equally suitable depending upon the particular application and use. The unitary container is preferably formed with a plurality of or at least three unsealable, sealed, and possible resealable receptacles wherein one or more or at least one of the three receptacles is formed with at least one receptacle unsealing scission element. In these variations, one or more of the receptacles may be preferably adapted to contain a substantially liquid substance. Another of the plurality of receptacles may preferably be being configured to contain a substantially powdered substance. Any of the receptacles can also be adapted to receive and contain and to be fillable with a substantially flowable substance that can be a powder, a liquid, and any combination of substances and materials having such powders and liquids thereon. And, a third receptacle of the plurality of receptacles can be being configured to receive a sheet material, which sheet materials can be formed as generally folded or flat items that can include sanitary latex or similar gloves, a disinfecting and prewetted wipe or towelette, an unfoldable changing mat, a bag having a drawstring that can receive a soiled diaper and be sealed, and any combination of similarly capable elements, components, and multiple-use items. Such multiple use items can include, for purposes of illustration and example but not for limitation, an unfolding mat sheet type material that can also be configured with in interior recess formed to function as a bag that can be used for the soiled diapers and other used items to be disposed of once the diaper is changed.

As described in connection with other embodiments and configurations, this next embodiments and configuration of the diaper changing kit of the instant invention can also incorporate one or more generally unitary containers that is or are releasably affixed to the exterior surface of the anterior portion of the diaper. The container can be affixed whereby a releasable adhesive and or fastener capability is incorporate that releasably holds the unitary container in position against the diaper until it is desired to be removed. Additionally, the releasable adhesive and or fastener is preferably selected so as to hold the container in place when exposed to the movements typically encountered by an active infant, child, or other diaper wearer during the process of changing the diaper. Preferably such an adhesive material or substance would be non-staining and would be fully removed as part of and with the unitary container after the diaper has been changed. In this way, the clothing and other garments of the person donning the diaper would not be exposed to any such adhesive substance or substances so as to avoid any staining or discoloration of such clothing or garments that may be donned after the new diaper is fastened. In addition to any these contemplated arrangements, the unitary container can also be formed with one or more adhesive areas or strips that may be fabricated and delivered to the retail user with a release liner material that covers and protects the releasable adhesive so that the purchaser or user of the inventive diaper kit may affix the unitary container to the diaper as may be desired by the user.

In yet other contemplated improved embodiments, the unitary container may also incorporate the described receptacles being fabricated so as to be fillable but where one or more of the contemplated plurality or at least three such receptacles is delivered to the purchaser or user empty so that the empty receptacle can be filled with a substance chosen by the user or purchaser of the contemplated diaper changing kit.

These variations, modifications, configurations, alternative arrangements, and alterations of the various preferred embodiments may be used either alone or in combination with one another as can be better understood by those with skill in the art with reference to the following detailed description of the preferred embodiments and the accompanying figures and drawings.

### BRIEF DESCRIPTION OF THE DRAWINGS

Without limiting the scope of the present invention as claimed below and referring now to the drawings and figures, wherein like reference numerals, and like numerals with primes, across the several drawings, figures, and views refer to identical, corresponding, or equivalent elements, components, features, and parts:

FIG. 1 is an elevated perspective view, in modified scale, of a diaper changing kit according to the principles of the instant invention;

FIG. 2 is an anterior view, in enlarged scale and rotated, of the diaper changing kit of FIG. 1;

FIG. 3 is superior view, in enlarged scale and rotated, of the diaper changing kit of FIGS. 1 and 2;

FIG. 4 is a cross-sectional view taken about section line 4-4 of FIG. 2, in enlarged scale and rotated, of the diaper changing kit of FIG. 2;

FIG. 5 is an anterior view, in modified scale, of a variation of a diaper changing kit according to the principles of the instant invention;

FIG. 6 is an anterior view, also in modified scale, of another variation of a diaper changing kit of the instant invention; and

FIG. 7 is also an anterior view, in modified scale, of yet another modification of a diaper changing kit according to the principles of the instant invention.

Also, in the various figures and drawings, various reference symbols and letters may be used to identify significant features, dimensions, objects, and arrangements of elements described herein below in connection with the several figures and illustrations.

### DESCRIPTION OF THE PREFERRED EMBODIMENT

In a wide range of possible embodiments and modifications and variations thereof, the diaper changing kit according to the principles, features, and capabilities of the instant inventive and novel arrangements establishes new and innovative solutions to the many problems that have long-plagued the art of changing single-use and reusable diapers, loincloths, napkins, and similarly capable undergarments. Even more specifically, the diaper changing devices, apparatuses, and kits described and contemplated herein are compatible for use with any of the wide range of prior art undergarments and diapers and can be custom-supplied by manufacturers at significant material and labor savings with optimized convenience for the intended users and with further capabilities for end-user or consumer customization, as is described in more detail herein elsewhere. Moreover, such capabilities for far-reaching compatibility with existing diapers, loincloths, napkins, and similarly capable undergarments vastly extend the possible uses and adaptations of the instant invention to so many more arrangements and configurations than could possibly be set forth in the exemplary disclosures set forth here.

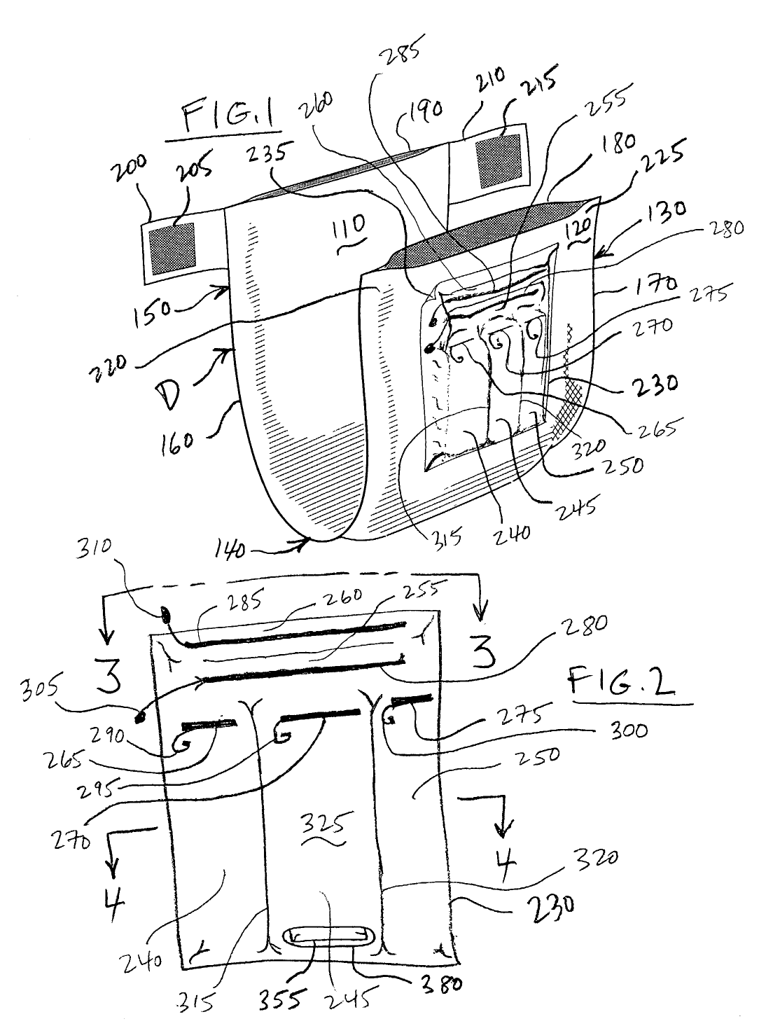

With reference now to the various and particularly to FIG. 1, a diaper changing kit 100 is depicted that includes an optional or preferable diaper, napkin, undergarment, or loincloth identified generally by reference letter “D.” As known to those skilled in the related arts, the undergarment or diaper D can be formed to incorporate interior and exterior surfaces 110, 120 that sandwich an absorbent material having a substantially liquid impermeable layer proximate the exterior surface 120 and that cooperate to define a substantially anterior or front facing portion 130 that is connected by an inferior perineal portion 140 to a generally posterior or rear portion 150.

In variations of the exemplary configuration, the undergarment, napkin, or diaper D can be further adapted to incorporate elastic materials and elements about dextral and sinistral lateral edges 160, 170 and or about anterior and posterior edges 180, 190. In any of a number of equally effective arrangements, the preferred or optional diaper or napkin or undergarment D can be further adapted with one of more fasteners 200, 210 that can improve the capability for fastening about a waist of the individual wearing the diaper or other similarly capable article D. Such fasteners 200, 210, such as those shown in FIG. 1 can be flexible and elastic and can be further selected to be ties, snaps, button and button-hole, adhesives, and as shown can be in the form of what are commonly referred to by those having skills in the related arts as flap-mounted hook and loop type fastening components 205, 215 that can releasably or permanently attach to corresponding attach portions 220, 225 on the exterior surface 120 of the anterior portion 130.

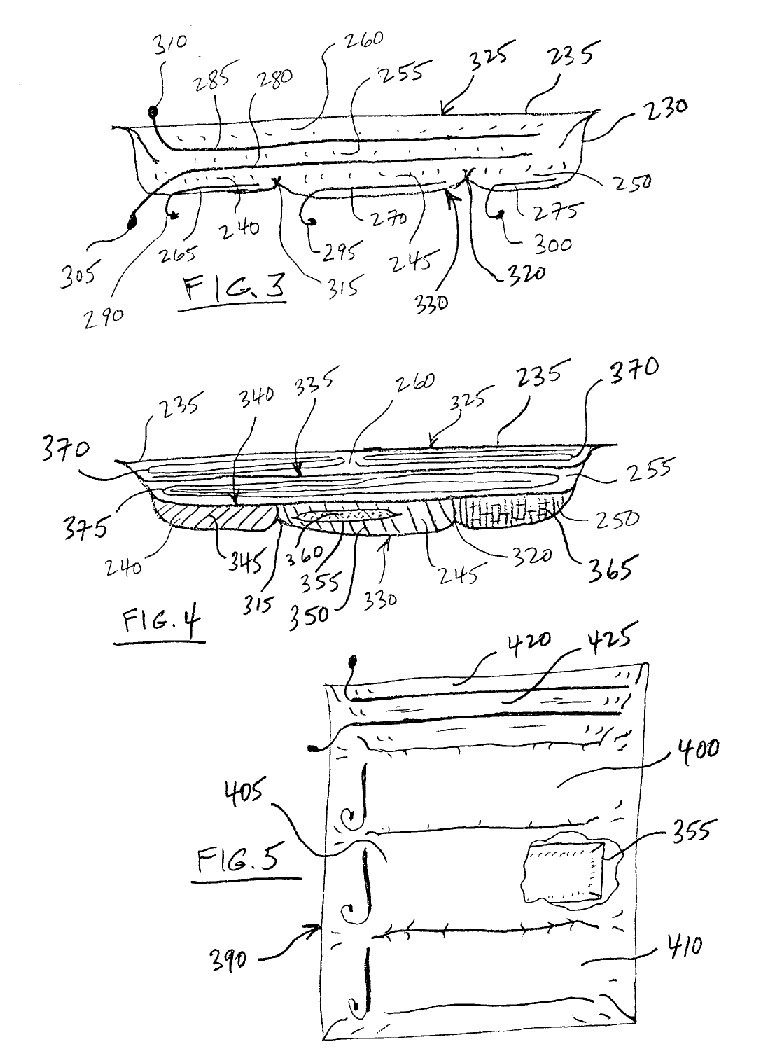

With continued reference to FIG. 1 and now also concurrently to FIGS. 2, 3, and 4, those with knowledge in the related and relevant arts may be able to further comprehend that the diaper changing kit 100 additionally incorporates one or more generally unitary or compartmented containers, such as container 230. The container 230 is adapted with an adhesive or other fastening capability such as hook and loop type fastening means about a generally posteriorly or aft facing surface 235 (FIGS. 1, 3 & 4) that is adapted to be affixed to the diaper, napkin, or undergarment D either optionally or preferably in permanently or releasably arrangements. In the example of baby or infant single-use diapers, such as those available from Proctor and Gamble under the American brand name “Pampers® Premium” or similar diapers, hook and loop type fasteners, particularly the hook portion thereof, have been demonstrated to work especially well in establishing a releasable fastening capability to the posteriorly or rear facing side of the container 230. Additionally one or more adhesives may be used that can be implemented either alone or in combination with any of the other attachment methods disclosed and contemplated herein. Such an adhesive can be selected to be a releasable adhesive that is applied to the aft or posteriorly facing portion of the container 230 and which is adapted to be completely removed from the diaper after the diaper change is completed and when the container 230 is removed therefrom. Upon removal, it is preferred that any such releasable adhesive be completely removed as part of the container 230 and that it leave no residue on the diaper surface that could stain or otherwise discolor have any other undesirable effect on the garments or clothing of the individual wearing the diaper.

The preferred container 230 is also preferably formed with one or more optionally or preferably sealed and unsealable pouches, compartments, or receptacles 240, 245, 250, 255, 260. One or all of the pouches, compartments, or receptacles 240, 245, 250, 255, 260 can be further adapted with one or more scission elements 265, 270, 275, 280, 285 that correspond to and are positioned and configured to unseal the respective pouches, compartments, or receptacles 240, 245, 250, 255, 260 on which it is formed or applied. Depending upon the particularly desired capability thereof, any or all of the respective scission elements 265, 270, 275, 280, 285 may also further incorporate a corresponding distally positioned pull tab 290, 295, 300, 305, 310 to improve the capability to grip and actuate the scission element 265, 270, 275, 280, 285. Each of the pouches, compartments, or receptacles 240, 245, 250, 255, 260 can be formed as separately sealed, unsealable, and resealable interstices that are defined by one or more side walls formed from seams 315, 320 and as sandwiched walls 325, 330, 335, 340. Any of a significant number of materials and seam forming techniques can be used that are compatible and well suited to the purposes and capabilities of the instant invention.

More specifically, the container 230 according to the principles of the instant invention can be formed from flexible sheets of metal foils, plasticized foils, metallicized plastic sheet, thermoset and elastomer monomers and polymers, and monomeric and polymeric thermoplastics including, for further purposes of illustration but not for purposes of limitation, thermoplastic materials selected from any of a variety of commercially available and suitable materials including plastic impregnated fabrics and clothes, acetal resins, fluorocarbons, polyesters, polyester elastomers, metallocenes, polyamides, nylon, polyvinyl chloride, polybutadienes, silicone resins, polypropylene, polyethylene, liquid crystal polymers, in woven fiber form and in alloys and combinations and mixtures and composites thereof, and reinforced alloys and combinations and mixtures and composites thereof. Each of such preferably and or optional materials can be joined with adhesives, thermowelding, ultrasonic welding, to name a few joining methods that are suitable for establishing the sandwiched walls 325, 330, 335, 340 and seams 315, 320.

In any of all of the preceding embodiments, variations, and alternative configurations thereof, the contemplated pouches, receptacles, and compartments 240, 245, 250, 255, 260 of the container 230 can be especially adapted to received and store substantially flowable substances including for purposes of example without limitation, powders, liquids, and combinations thereof, as well as to contain and store packets of such substantially flowable substances. Substantially flowable substances that are well-suited for use in the context of the instant invention include, for further illustrative purposes but not for purposes of limitation, baby oil, lotions, aloe vera type and other gels, talcum powder and similarly configured powders, various medicaments including rash ameliorating and soothing substances and balms. In addition to receiving, containing, storing, and dispensing such substantially flowable substances, the pouches, compartments, and receptacles 240, 245, 250, 255, 260 of the container 230 are also readily adapted to receive, store, and dispense any number of substantially flat items or items that can be flattened for receipt into the receptacles, such as latex or silicone type sanitary and protective gloves, and other types of sheet materials that can be used for cleaning, wiping, disinfecting, and toweling during the diaper change procedure. Including gloves in the contemplated diaper changing kit according to the principles of the instant invention can be especially desirable by healthcare workers in view of the widely practiced “universal precautions” that have been implemented in health care and hospice care type environments, which precautions urge, recommend, and in some instances, require the use of one or more pairs of gloves to be worn by the worker whenever exposure to human bodily fluids and waste is contemplated, possible, probable, or likely.

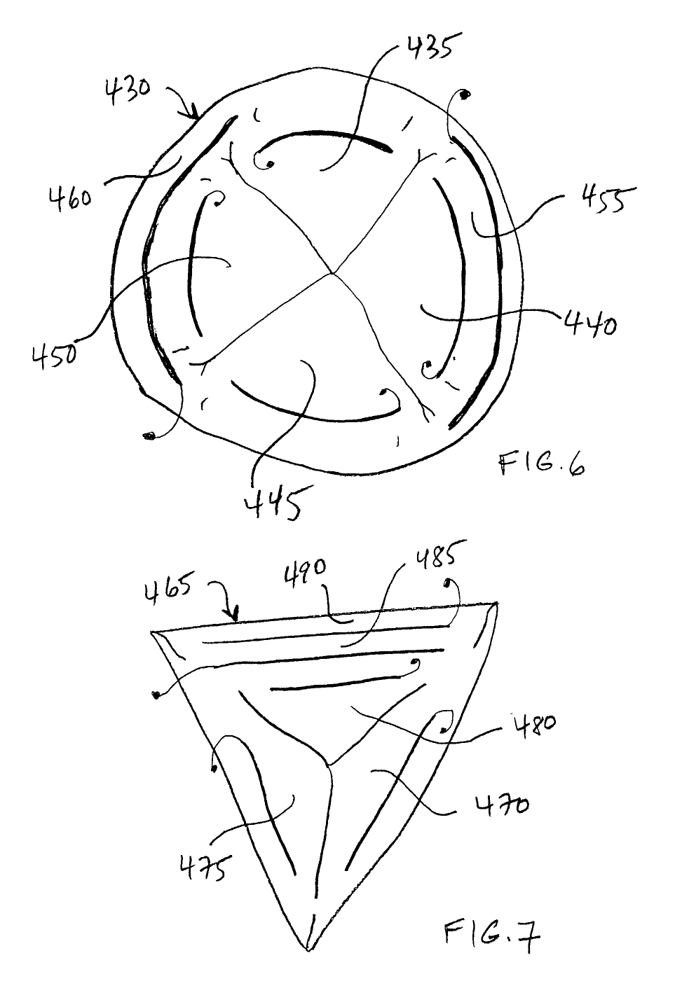

More specifically, and with specific reference to FIG. 4 as well as the other various illustrations, each pouch, compartment, or receptacle 240, 245, 250, 255, 260 can receive, contain, store, and dispense such substantially flowable substances and wiping materials wherein compartment or receptacle 240 can be filled with a lotion 345. Another of the compartments such as receptacle 245 can be filled with a powder such as talcum-type baby powder 350. In additional alternative variations, receptacle or compartment 245 can also additionally or alternatively receive and store a packet or pouch 355 filled with a balm or gel substance such as an aloe lotion or other medicament 360. In further modifications and variations, receptacle or compartment 250 can receive, store, and dispense another substantially flowable substance such as a dermally therapeutic agent such as a steroidal cream 365. Such contemplated substantially flowable substances can be dispensed from their respective pouches, compartments, or receptacles 240, 245, 250, 255, 260 in the container 230 by opening and unsealing with the respective scission elements 265, 270, 275, 280, 285. In operation, the scission elements 265, 270, 275, 280, 285 are actuated by pulling the tabs, which severs the material forming the receptacles 240, 245, 250, 255, 260 to establish an opening through which the substantially flowable substance can be communicated. Various sized openings can be established by varying the length of the scission elements 265, 270, 275, 280, 285 and or by tearing the opening only so much as is needed to effect the desired dispensing opening. To maintain the efficacy of and extend the shelf-life of the substantially flowable substances, each of the pouches, compartments, or receptacles 240, 245, 250, 255, 260 can be further adapted whereby the interior surfaces can be coated or otherwise treated to add further sealing properties thereto.

The diaper changing kit 100 also further contemplates embodiments wherein the pouches, compartments, or receptacles 255, 260 are especially adapted to receive, store, and dispense folded or otherwise generally flat items configured as wiping and cleaning sheet materials such as one or more folded pre-wetted wipes 370 and a folded storage and or disposal bag 375 and combinations thereof. In alternative arrangements, the receptacle or compartment 260 can be sized and adapted, such as to have an expandable accordion style capability to be a sealable container for capturing, containing, and disposing of soiled items including a disposable diaper. In configurations of any of the preceding embodiments, configurations, and alternative arrangements, the sealable container for receiving a soiled diaper and similarly used and soiled cleaning items can be further adapted with closure elements that can include one or more components such as a draw-string, a cinch-cord, and or an adhesive strip and release liner capability, which liner can be removed to expose the adhesive, and any of which closure elements can be used alone or in combination to seal and close the compartment that is received with the soiled items.

With continued reference to the various figures and with specific reference again to FIGS. 2 and 4, those skilled in the art may further by able to understand that one or more of the pouches, compartments, or receptacles 240, 245, 250, 255, 260 in the container 230 can incorporate in place of or in combination with the scission elements 265, 270, 275, 280, 285 one or more apertures or openings, such as opening or aperture 280, defined or formed in the container 230 to be in communication with the respective unsealed pouch, compartment, or receptacle 240, 245, 250, 255, 260. In this alternative or preferred configuration, a sealed packet or pouch can be inserted, stored, and retrieved from the respective receptacle or compartment 245 to dispense the substantially flowable substance 360 contained in the packet or pouch 355.

Each or any of the scission elements 265, 270, 275, 280, 285 can be formed as a rip-cord type severing cord that operates much like a tear strand or wire that is adapted to sever the material that forms the wall of the pouch, receptacle, or compartment 240, 245, 250, 255, 260 much like a tear off tab that forms a part of a convenient opening means of a small package of an American-style chewing gum wrapper or the similarly constructed opening means of a cellophane plastic wrapping that is customarily employed by manufacturers to protects a new, unused CD-ROM jewel case. In variations of the described configurations, the scission elements 265, 270, 275, 280, 285 can be a tongue and groove sliding zipper-like resealable device similar in construction and function to those used in resealable plastic polypropylene bags commonly available from S.C. Johnson and Sons Company, USA, under the brand name of ZIPLOC® bags. In the resealable configurations, the pouch, receptacle, or compartment 240, 245, 250, 255, 260 can be custom-filled either by a pharmacist seeking to fill a medical prescription, or by care giver or parent seeking to have a portable supply of preferred substantially flowable substances available for diaper changes away from home or the care giving facility or location. Additionally, such resealable configurations can be useful for retaining unused portions of such flowable substances for later use to minimize waste.

Referring now also to FIGS. 5, 6, and 7, those skilled in the relevant arts may be able to further appreciate that the preferred, modified, and alternative arrangements of any of the preceding embodiments and variations thereof can also incorporate any number of equally suitable arrangements of the various elements, components, and features of the contemplated diaper changing kits. More specifically, although a substantially rectangular shape is illustrated in FIGS. 1 through 4 for the container 230 and even though substantially inferior to superior longitudinal orientations of the pouches and receptacles 240, 245, 250, 255, 260 are shown, any number of alternative arrangements can be easily implemented. For purposes of further examples but not for purposes of limitation, in FIG. 5 a modified container 390 is shown to have substantially lateral pouches, receptacles, or compartments 400, 405, and 410 are depicted as being incorporated with the compartments 415, 420 that are configured similarly to compartments 255, 260. A cut-away portion of compartment 405 reveals packet or pouch 355 received therein.

In FIG. 6, another variation is described wherein a substantially circular container 430 incorporates wedged-shaped unsealable and optionally resealable pouches, receptacles, or compartments 435, 440, 445, 450, 455, 460 are respective scission elements similarly configured to those of earlier described embodiments. In FIG. 7, substantially wedge-shaped container 465 is depicted formed with unsealable and optionally resealable compartments, receptacles, or pouches 470, 475, 480, 485, 490.

Numerous alterations, modifications, and variations of the preferred embodiments disclosed herein would be apparent to those skilled in the art and they are all contemplated to be within the spirit and scope of the instant invention, which is limited only by the following claims. For example, although specific embodiments have been described in detail, those with skill in the art can understand that the preceding embodiments and variations can be modified to incorporate various types of substitute and/or additional materials, relative arrangement of elements, and dimensional configurations for compatibility with the wide variety of possible diapers, loincloths, napkins, and sanitary napkins that are available in the marketplace. Accordingly, even though only few such embodiments, alternatives, variations, and modifications of the present invention are described herein, it is to be understood that the practice of such additional modifications and variations and the equivalents thereof, are within the spirit and scope of the invention as defined in the following claims.

1. A self-contained diaper changing kit, comprising:
a diaper having interior and exterior surfaces and formed with a substantially anterior portion connected by an inferior perineal portion to a generally posterior portion; and
a generally unitary container attached proximate to the exterior surface of the anterior portion and being formed with a plurality of unsealable receptacles with at least one, receptacle of the plurality being formed with at least one scission element adapted to unseal the at least one receptacle.

2. The self-contained diaper changing kit according to claim 1, wherein the generally unitary container is releasably affixed to the exterior surface of the anterior portion of the diaper.

3. The self-contained diaper changing kit according to claim 1, wherein the generally unitary container includes at least one of the plurality of unsealable receptacles being received with a substantially flowable substance.

4. The self-contained diaper changing kit according to claim 1, wherein the generally unitary container includes at least one of the plurality of unsealable receptacles being received with a sealed packet that is adapted to be filled with a substantially flowable substance.

5. The self-contained diaper changing kit according to claim 1, wherein the generally unitary container further incorporates at least one aperture formed in the container and in communication with an unsealed receptacle of the plurality being received with a sealed packet that is fillable with a substantially flowable substance.

6. The self-contained diaper changing kit according to claim 1, wherein the scission element of the generally unitary container is formed as a severing tear strip.

7. The self-contained diaper changing kit according to claim 1, wherein the scission element of the generally unitary container is formed as a severing rip cord having a distal pull tab.

8. The self-contained diaper changing kit according to claim 1, wherein the scission element of the generally unitary container is formed as a substantially flexible and resealable tongue and groove sliding fastener.

9. A self-contained diaper changing kit, comprising:
a diaper having interior and exterior surfaces and formed with a substantially anterior portion connected by an inferior perineal portion to a generally posterior portion; and
a generally unitary container attached proximate to the exterior surface of the anterior portion and being formed with at least three unsealable receptacles with at least one of the three receptacles being formed with at least one receptacle unsealing scission element and with one receptacle adapted to contain a substantially liquid substance, with another receptacle being configured to contain a substantially powdered substance, and a third receptacle being configured to receive a sheet material.

10. The self-contained diaper changing kit according to claim 9, wherein the generally unitary container is releasably affixed to the exterior surface of the anterior portion of the diaper.

11. The self-contained diaper changing kit according to claim 9, wherein the generally unitary container includes at least one of the three unsealable receptacles being received with a sealed packet that is adapted to be filled with a substantially flowable substance.

12. The self-contained diaper changing kit according to claim 9, wherein the generally unitary container further incorporates at least one aperture formed in the container to be in communication with an unsealed receptacle of the at least three receptacles being received with a packet that is fillable with a substantially flowable substance.

13. The self-contained diaper changing kit according to claim 9, wherein the scission element of the generally unitary container is formed as a severing tear strip.

14. The self-contained diaper changing kit according to claim 9, wherein the scission element of the generally unitary container is formed as a severing rip cord having a distal pull tab.

15. The self-contained diaper changing kit according to claim 9, wherein the scission element of the generally unitary container is formed as a substantially flexible and resealable tongue and groove sliding fastener.

16. A self-contained diaper changing kit, comprising:
a generally unitary container formed with a substantially planar attach surface that is coated with a fastening material adapted to affix the container to an exterior surface of a diaper, the container being formed with at least three unsealable receptacles with at least one of the three receptacles being formed with at least one receptacle unsealing scission element and with one receptacle adapted to contain a substantially liquid substance, with another receptacle being configured to contain a substantially powdered substance, and a third receptacle being configured to receive a sheet material.

17. The self-contained diaper changing kit according to claim 16, wherein the unsealable receptacles are resealable.

18. The self-contained diaper changing kit according to claim 16, wherein the generally unitary container includes at least one of the three unsealable receptacles being received with a sealed packet that is adapted to be filled with a substantially flowable substance.

19. The self-contained diaper changing kit according to claim 16, wherein the generally unitary container further incorporates at least one aperture formed in the container to be in communication with an unsealed receptacle of the at least three receptacles being received with a packet that is tillable with a substantially flowable substance.

20. The self-contained diaper changing kit according to claim 16, wherein the scission element of the generally unitary container is formed as a severing tear strip.

21. The self-contained diaper changing kit according to claim 16, wherein the scission element of the generally unitary container is formed as a severing rip cord having a distal pull tab.

22. The self-contained diaper changing kit according to claim 16, wherein the scission element of the generally unitary container is formed as a substantially flexible and resealable tongue and groove sliding fastener.
